(19) United States
(12) Patent Application Publication
(10) Pub. No.: US 2010/0304421 A1
(43) Pub. Date: Dec. 2, 2010

(54) OPTICAL MONITORING METHOD

(75) Inventors: Sergey Anatoliyovich Piletsky, Cranfield (GB); Igor Meglinski, Cranfield (GB); Ewa Moczko, Srebrna Gora (PL)

Correspondence Address: STITES & HARBISON PLLC, 1199 NORTH FAIRFAX STREET, SUITE 900, ALEXANDRIA, VA, 22314, US

(73) Assignee: CRANFIELD UNIVERSITY, Cranfield, Bedfordshire (GB)

(21) Appl. No.: 12/681,217

(22) PCT Filed: Oct. 6, 2008
(86) PCT No.: PCT/GB2008/003389

§ 371 Date: Aug. 20, 2010

(30) Foreign Application Priority Data
Oct. 4, 2007 (GB) 0719373.3

(51) Int. Cl. C12Q 1/02; G06N 5/02; G06N 3/02

(52) U.S. Cl. 435/29; 706/54; 706/20

(57) ABSTRACT

A mixture of dyes each having an optical property (e.g. fluorescence intensity) which is sensitive to an external factor is caused to interact with a system that is to be monitored, such that the system provides the external factor and therefore influences the optical properties of the dyes. The optical properties of the individual dyes are individually measurable (e.g. being spectral intensities at different wavelengths). Their values are subjected to pattern analysis, e.g. using an artificial neural network analysis, leading to an output that characterises the system or its state.

### TECHNICAL FIELD

The present invention relates to an optical method for analysis and monitoring. It may be used for the detection and monitoring of various changes in physico-chemical parameters, concentration of analytes and monitoring chemical and biological processes.

### BACKGROUND ART

There is a considerable interest in developing sensor arrays that can measure multitude of chemical and physical parameters. Some of these devices are not aimed at measuring precise concentrations of analyte, but rather trend, tendency or pattern characteristic for chemical or biological (physiological) processes. Typically these devices are electrochemical, surface acoustic wave (SAW) or bulk acoustic wave (BAW) sensors which are capable of identifying patterns from the collected signals, acting as mimic of a human “electronic nose” or “electronic tongue” (U.S. Pat. No. 6,350,369 and U.S. Pat. No. 7,144,553). While the concept of pattern recognition as an approach for detection of analytes has been explored in electrochemical or acoustic sensors, only limited efforts were put into developing similar optical sensor or optical sensing system (see e.g. U.S. Pat. No. 5,512,490). In existing examples however this principle was applied in combination with spatially discrete sensor elements. The main focus of the present invention is development of optical equivalent of “electronic nose” using mixture of dyes.

Fluorescence spectroscopy has proven to be a useful tool for multiplex measurements of analytes and different parameters in environmental and biological samples. The technique appears to be the most promising because of its high sensitivity, minimal-invasive measurements, ability to monitor several parameters simultaneously with a single instrument and to detect real-time responses in a target. An additional advantage is that it is a fast and low cost method.

The method of using fluorescent particles for multiplexed measurements of analytes is described in U.S. Pat. No. 7,141,431. Chandler et al. disclose in this invention the technique for preparing polymeric particles stained with different fluorescent dyes. The purpose of staining is to produce particles with unique fluorescent signatures. These signatures are used for discriminating particles coupled with different biological material, such as antigens, antibodies, enzymes or nucleic acids. The interactions of particles with analytes are monitored by flow cytometry and electrophoresis. It would not be recommended however using environment-sensitive dyes as labels here since their varying spectra will interfere with particles identification. Yet another invention, U.S. Pat. No. 6,049,380 describes an application of discrete fluorescent dyes for analysis of characteristics of single molecule. U.S. Pat. No. 5,512,490 describes an optic array formed of multiple semi-selective sensing receptors immobilised at different spatial positions. U.S. Pat. No. 5,747,349 describes fluorescent beads with an array of environment-sensitive dyes suitable for application in blood analysis by flow cytometry. The use of optical signatures for identification, tracking and categorization of the components of different mixtures was disclosed also by Nova et. al. in U.S. Pat. No. 6,319,668. It includes preparing a library of synthesized compounds comprising molecules or biological particles linked to a solid support matrix. All information is coded, e.g. as optically-readable symbols, called bar-codes, and stored as matrices with memories.

A collection of discrete environment-sensitive dyes have been used by Caputo, et. al. in U.S. Pat. No. 5,830,134. They describe a mechanism for determining and monitoring the value of at least one physico-chemical parameter of a medium, such as pO, pCO or pH of human body fluids. The detection and monitoring of these parameters is possible by using a sensitive medium with immobilised chromophores with unique optical properties. Identification of a particular parameter is obtained by pattern recognition via comparing monitored profile with predetermined profile stored in the data base entered in the memory. Yet another array of discreet optical sensor elements has been used by Kauer in U.S. Pat. No. 6,649,416 for detecting and discriminating wide variety of target analytes (odours, i.e. vapour analytes) in complex sample mixtures. The optical response signal of the array to the odour is detected and converted to an electrical voltage signal. An analyte identification algorithm compares measured optical responses to characteristic optical responses of sensor elements and matches the result to known target odours. An artificial neural network has been used by Ham, et. al., in U.S. Pat. No. 5,553,616 for the detection of glucose or other biological substances in complex biological samples.

Several different luminescent cell reporters were used for monitoring of presence of toxin as described in U.S. Pat. No. 7,160,687. The approach described there relies on cells ability to react to toxin by changing their metabolism, and as a result, altering the luminescence of specific reporter molecules, such as microtubule-associated proteins, actin, or actin-binding proteins. No pattern analysis has been applied in this work.

### DISCLOSURE OF INVENTION

We have appreciated that it would be advantageous to provide a sensor system which is based not on an array of spatially discrete physical detectors, but on a mixture of dyes with each dye representing a different sensing element. Such a system would allow cost-effective detection of various analytes in a small volume of sample. The present invention describes application of a mixture of at least two environment-sensitive dyes which can provide optical responses (e.g. change in absorbance, transmission, reflectance, emission or luminescence) to change in the environment (e.g. variations in pH, temperature, ionic strength, solvent polarity, presence of redox species, surfactants, oxygen, organic, inorganic and biological species). Such a sensor system may be usable as a disposable reagent having corresponding advantages such as easy application and low price of analysis.

Thus the invention provides a method of monitoring a substrate comprising:

a) providing a mixture containing a plurality of dyes each having an optical property which is sensitive to an external factor, wherein said optical properties of the different dyes are individually monitorable;
b) enabling the mixture of dyes to interact with the substrate which provides said external factor, thereby influencing said optical properties of the dyes;
c) monitoring said optical properties of said plurality of dyes to provide a corresponding plurality of output signals; and
d) subjecting said plurality of output signals to a pattern recognition method, thereby to characterises the substrate or a parameter or condition thereof.

The present invention describes the analytical tools for chemical, biochemical and physico-chemical analysis. The idea is to use a mixture of environmental-sensitive dyes capable of generating an optical signal (such as change in absorbance, transmission, reflectance, emission or luminescence within UV, visible or IR regions) in response to possibly complex physical or chemical changes. Dyes are selected in accordance with their ability to contribute to a characteristic pattern of the mixture's spectral characteristics. The diversity of dyes selected is desirably sufficiently broad for allowing qualitative and quantitative detection of at least two or more chemical or physical parameters such as pH, temperature, pressure, and concentration of metabolites. The principle is similar in nature to so-called “electronic nose” sensors which employ a combination of sensor elements for analysis of complex mixtures. The advantage of the proposed approach lies in greater flexibility and possibility for miniaturization. Thus small drop of dyes can be applied locally and analyzed using an external optical sensor.

A preferred embodiment of the invention will now be described with reference to the accompanying drawings,

### BRIEF DESCRIPTION OF DRAWINGS

FIG. 1 shows three-dimensional color mapped surface diagram of 5 fluorescent dyes; FIG. 1′ is a monochrome copy.

FIG. 2 shows change in fluorescence intensity of dyes mixture as a function of pH (A); and a linear regression between the experimental and predicted values of pH made using artificial neural network (B).

FIG. 3 shows change in fluorescence intensity of dyes mixture as a function of temperature (A); and a linear regression between the experimental and predicted values of temperature made using artificial neural network (B).

FIG. 4 shows change in fluorescence intensity of dyes mixture added to growing cell culture as a function of incubation time (A); and a relationship between real and simulated time of cell culture growth using artificial neural (B).

FIG. 5 shows application of PCA for differentiating two yeast strains using fluorescent data obtained from the mixture of five fluorescent dyes.

FIG. 6 is a set of graphs showing correlation between actual (measured) and determined by ANN values of pH (A), temperature (B), dissolved oxygen (DO) concentration (C) and PBS concentration (D).

### MODES FOR CARRYING OUT THE INVENTION

The present invention employs a mixture (array) of different dyes capable of generating an optical signal related to physical and/or chemical changes in the environment. The “optical signal” is generally defined as values or change in values of absorbance, transmission, reflectance, scattering, emission (e.g. luminescence or fluorescence) within UV, visible or IR regions. The dye “sensitivity” is defined as an ability to change an optical property (e.g. fluorescence intensity, decay time or spectrum) in response to change in its environment, possibly as a result of interaction with analyte. We may provide dyes with sensitivity to changes in pH, temperature, ionic strength, solvent polarity, presence of redox species, surfactants, oxygen, organic, inorganic and biological species. A number of dyes suitable for this purpose is described in the art (see e.g. R. P. Haugland, Handbook of Fluorescent Probes and Research Chemicals, 5th Edition, Molecular Probes Inc., Eugene, 1992). The examples include oxygen sensitive dye such as ruthenium(II)-tris-(4,7-diphenyl-1,10-phenanthroline)-diperchlorate, pH-sensitive dye Carboxy SNAFL-1 (Molecular probes) etc. Dyes might react with analytes, forming physical or chemical bonds. The selection of dyes is performed in accordance with two criteria: (i) dyes should have an ability to change optical properties in response to change in environment, and (ii) it should be possible to monitor at least some contribution from the individual dyes in the total spectrum of dye mixture. Desirably use is made of a dye array containing two or more, preferably five or more, dyes. Preferably, dyes are fluorescent compounds, although non-fluorescent species can be used as well in some embodiments of the present invention. Dyes may exhibit light emission at a wavelength in the ultra-violet, visible range or near infrared region, preferably greater than about 400 nm. The selected dyes should possess substantially different emission spectra, preferably having adsorption or emission maxima separated by greater than 10 nm. The spectral characteristics of dye and changes caused by interactions with their surroundings can have linear or non-linear relationship. In one type of embodiment of the present invention, one or more dyes having no particular sensitivity to changes in the environment are used as standards.

We may use dyes which are soluble in aqueous environment, organic solvent or mixed solvent. We may use dyes which are distributed in gels or in polymers. The examples of dyes which may be used in the present invention include, but are not restricted to, compounds and derivatives of: cyanine, quinine, squaric acid, cyclobutenedione, hydroxypyrene, acridine, cresol red, flavine, alizarin, indamine, coumarin, dansyl, eosin, rhodamine, nile red, nitrobenzoxadidole, ninhydrin, oregon green (TM), oxazine, malachite green, phthalocyanine, porphyrin, stilbene, texas red, methyl orange, or combinations thereof.

The invention provides generic analytical tools for chemical, biochemical, biophysical and physico-chemical analysis. A mixture of dyes may be used as a disposable reagent added to a solution to be analysed. A mixture of dyes may be used as a component of a sensor, or of an indicator or marker. A mixture of dyes may be used for control of chemical or biological processes. In one type of embodiment of the present invention a mixture of dyes is added to a growing cell culture and used for monitoring, for example development phases, change in pH, and/or concentration of nutrients.

In other embodiments, mixture of dyes may be used for a precise measurement of values of pH, temperature, ionic strength, solvent polarity, presence of redox species, surfactants, oxygen, organic, inorganic and biological species. To achieve this, the optical signals of dye mixture in an appropriate model or real conditions may be calibrated by adding aliquots of corresponding analytes or by varying corresponding physical parameters. Mixtures of dyes may also be used for identification of specific analytes and mixtures of different analytes by reference to and comparison with previously established spectral recognition patterns for a variety of known chemical compounds and compositions. The preferable mode for using mixtures of dyes is for monitoring changes in concentrations of analytes or physical parameters or monitoring an event. Dye mixtures may also be deposited onto the skin for monitoring physiological parameters of living organisms including humans. Dye mixtures, may also be deposited onto a sensor surface for use in in vivo monitoring of physiological parameters of living organisms including humans. Dye mixtures may be used for the identification of abnormal status in tissues and for diagnosis of diseases or abnormal physiological status. Dye mixtures may be used for screening purposes. Dye mixtures may be used to monitor toxic effects or biological effects of drugs or drug candidates.

Dye mixtures may be used for identification of tampering with materials, articles, and commercial products. Dye mixtures may be used as an indicator of quality, e.g. for monitoring quality of food. Dye mixtures may be used for monitoring of ageing processes of materials and commercial products. Dye mixtures may be used for monitoring or exposure to radiation including, but not restricted to, visible light, UV, X-ray, electrons and particles.

Dye mixtures may be deposited in spatially different positions for monitoring of spatial or temporal changes in the environment. Dye mixtures may be used for detecting pollutants in air, water, and soil. Dye mixtures may be used for qualitative and quantitative measurement of analytes and gases in the blood.

The present invention involves an, analysis of the optical response of a mixture of dyes. We may analyse the optical signal from a dye mixture by measuring its spectrum before and after a change occurs and comparing values of UV/visible/IR absorbance, transmission, reflectance, scattering or fluorescence/luminescence emission. In some embodiments the optical signal from a dye mixture is analysed by measuring whole spectrum of dyes. In other aspect of the invention only some spectral characteristics of dyes are analysed e.g. absorbance, emission or reflectance at particular wavelengths, such as peak maxima for corresponding dyes.

We may perform comparison of optical characteristics of dye mixture before, during and/or after a change occurs. We may perform comparison of optical characteristics of a dye mixture in a real sample where environmental changes occur and also in a control sample where the environment does not change.

We may perform comparison of optical characteristics of dyes sensitive to environment with those not sensitive to environment (standards or references). This approach will allow compensating for dilution effect and non-characteristic changes (e.g. turbidity) in the environment.

We generally evaluate data by calculation using an appropriate pattern recognition algorithm.

We may analyse the optical response as a pattern of spectral change progressions (measured at different wavelengths) which are monitored over time and are evaluated collectively as an assemblage of different spectral responses generated concurrently. The collective pattern is then used as the basis for recognition and identification of an analyte, mixture of different analytes or particular change in the environment. We may use a library of reference spectral recognition patterns as the means for evaluating and identifying the analyte, a mixture of analytes or characterise change in the environment. We may monitor changes in the environment by using equipment capable of measuring multiple excitation/emission wavelengths. The source of light may be an incandescent lamp, an arc or flash lamp, a solid state emitter, pulse or continuous wave laser. Preferably, the source of light is a light emitting diode. The optic sensing apparatus and instrumentation system detects changes in light intensity or changes in light wavelength over time. The light energy emitted from the dye mixture, is collected using any detector known in the art, e.g. photographic plate, photo-diode, CCD, CMOS, photomultiplier, avalanche photo-diode (APD), that are combined with an appropriate collecting optics (e.g. microscope, fibre optic, set of lenses, mirrors and diaphragms, and their various combinations etc.) and convenient image processing capabilities. A preferred optical detection system utilises a computerised digital signal processing unit capable of detecting, recording, and memorizing the individual spectral responses, as well as capable of generating collective patterns and the ability to employ and compare sets of recognition patterns in order to detect and identify analytes or characterise ongoing chemical, biochemical, physiological, physical, biophysical, mechanical and other relevant processes. After the measurement procedure the optical/NIR/fluorescent signal is transformed to the digital array. This array is used as an input data for further Principal Components Analysis, Multi-Regression Analysis, Cluster Analysis or Artificial Neural Network (ANN) or other pattern recognition methods known to specialists in art.

### Examples

The present invention will now be further described particularly with references to the following non-limiting examples.

### Example 1

### Preparation of Dye Mixture for Fluorescence Analysis

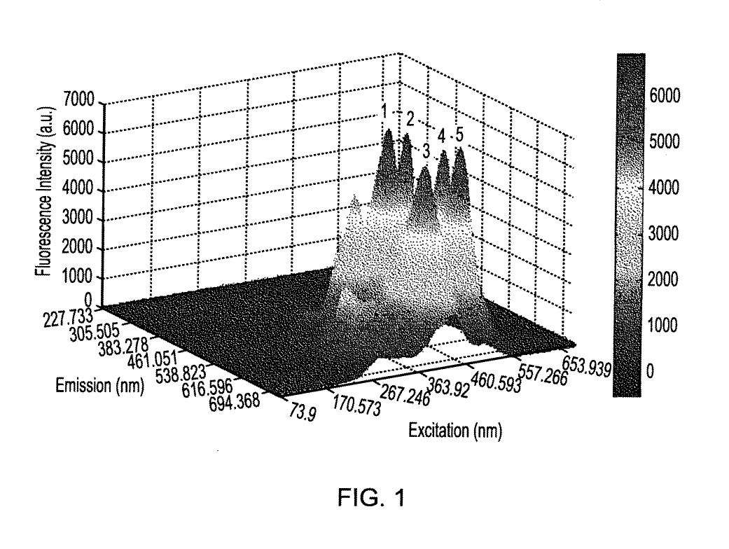

The stock solutions of following dyes were prepared in deionised water: dye 1: 0.15 mM 8-Hydroxypyrene-1′,3,6-Trisulfonic Acid; dye 2: 0.025 mM Oregon Green 514; dye 3: 0.1 mM Rhodamine B; dye 4: 6 mM Tris (4,7-diphenyl-1,10-phenanthroline) ruthenium dichloride; dye 5: 2 mM Thionin Acetate, and 200 μl aliquots of each dye were mixed together to produce the dye mixtures used in the following experiments. The fluorescence intensity measurements were performed using a three-dimensional spectrofluorimeter Jobin Yvon—SPEX FL-3D (Instruments SA, Stanmore, Middlesex, UK) at 0.5 s of time exposure. The spectra were recorded over a range of excitation (227.7-724.5 nm) and emission (73.9-691.4 nm) wavelengths and analysed using Grams/32 (version 4.14 Level II, Galactic Industries Corporation) and MATLAB (version 7.3.0.267 R2006b MathWorks Inc., 2006) softwares. The Grams software allows the data to be saved in *spc format and then using a suitable script in MATLAB they are converted into image format. The fluorescence measurements were performed in 3 ml of 50 mM PBS in 4 ml quartz cuvettes having a light path of 10 mm, with 0.5 s exposure times. The typical spectrum of dye mixture is presented in FIG. 1. The measurements of fluorescence intensities were taken using wavelengths presented in Table 1 and used in following the experiments.

TABLE 1
The excitation and emission wavelengths for mixed fluorescent
dyes used in experiments.

| Fluorescent Dye | Excitation [nm] | Emission [nm] |
|---|---|---|
| 8-Hydroxypyrene-1′,3,6-Trisulfonic | 480 | 527 |
| Acid | | |
| Rhodamine B | 545 | 576 |
| Thionin Acetate | 470 | 637 |
| Oregon Green 514 | 504 | 528 |
| Tris (4,7-diphenyl-1,10- | 545 | 628 |
| phenanthroline) ruthenium dichloride | | |

### Example 2

### pH Dependence of the Optical Properties of Dyes Mixture

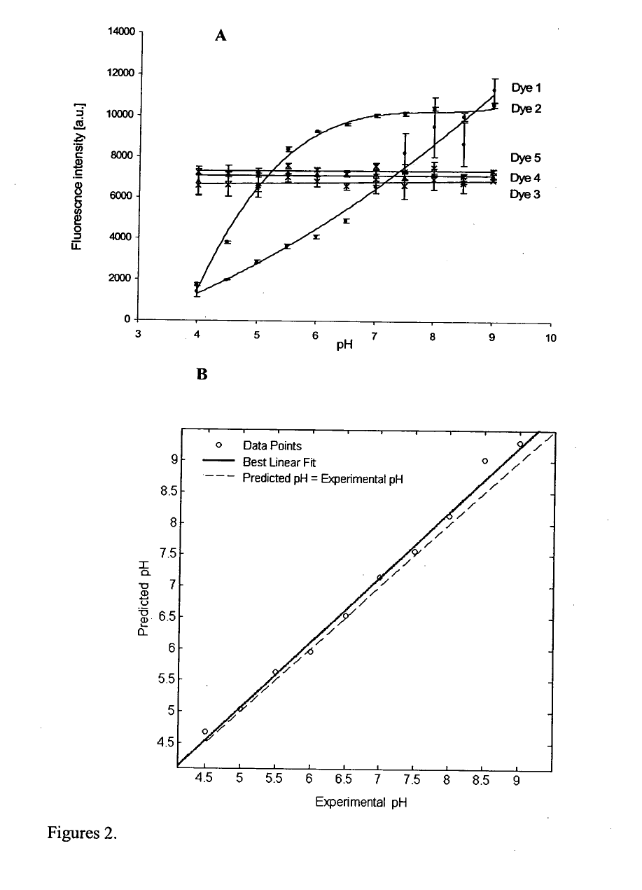

Fluorescent Measurements were Performed in 50 Mm Acetate, Phosphate and Borate buffers with pH ranging from pH 4.0 to pH 9.0. In each measurement 3 ml of suitable buffer is transferred into quartz cuvette and then 50 μl of the mixture of five fluorescent dyes is added. The pH dependence of the fluorescence intensity of the mixture of five dyes is shown in FIG. 2 A. The maximum of fluorescence intensity of each dye is plotted as a function of pH values.

The neural network structure used in this study consisted of a single linear neuron with five neurons in the input layer that corresponded to the intensities of 5 fluorescent dyes and one neuron in the output layer. The network is first trained by presentation of measured values of fluorescence intensities of dyes with desired output values (values of parameters, eg. pH, temperature). The training process involves adjusting internal ANN weights in a manner that will minimise the error between the input values and the corresponding desired output values by the Widrow-Hoff (LMS) learning algorithm (Demuth, H. & Beale, M., Neural Network Toolbox v4.0, 2003). Learning cycles update weights of output layer and progress backwards. The performance goal for the training of ANN is set as 0.01. Once the system is trained sufficiently, the neural network can distinguish different input data according to its learning experience. The regression analysis was performed on the predicted (simulated) values of parameters against the actual (measured) values. The results of simulation are shown in FIG. 2 B. This figure illustrates linear regression between measured values of parameters (Targets) and simulated, predicted values (Network Output).

### Example 3

### Temperature Dependence of the Optical Properties of Dye Mixture

The temperature-induced changes in fluorescence intensity of the dye mixture as used in Examples 1 and 2 were monitored and analysed in the similar way as described above. The temperature of the quartz cuvette containing the dye mixture in 3 ml of 50 mm PBS buffer was adjusted externally within range of 20-40° C. using thermostatic water-bath (Grant Instruments Ltd, Model 0331, Cambridge, UK).

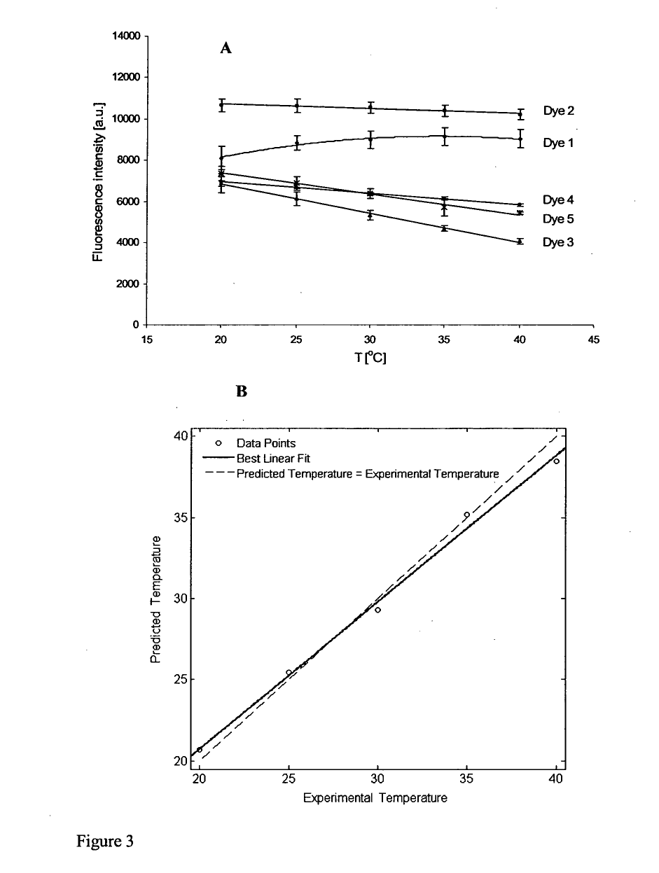

The temperature dependence of the fluorescence intensity on the mixture of five dyes is shown in FIG. 3 A. The maximum of fluorescence intensity of each dye is plotted as a function of temperature.

The relationship between the fluorescence intensity changes of the mixture and temperature values was investigated using artificial neural network as described above. The neural network structure used in this study consisted of a single linear neuron with five neurons in the input layer that corresponded to fluorescent emission of 5 fluorescent dyes (measured at wavelengths shown in Table 1) and one neuron in the output layer. The development of neural networks was implemented using MATLAB version 7.3.0 and the Neural Network Toolbox version 4.0.1. The neural network was trained and simulated with data from fluorescent measurements. The regression analysis was performed on the predicted (simulated) values of parameters against the actual (measured) values. The results of simulation are shown in FIG. 3 B. This figure illustrates linear regression between measured values of parameters (Targets) and simulated, predicted values (Network Output).

### Example 4

### Monitoring of Cell Growth Phase Using Mixture of Five Dyes

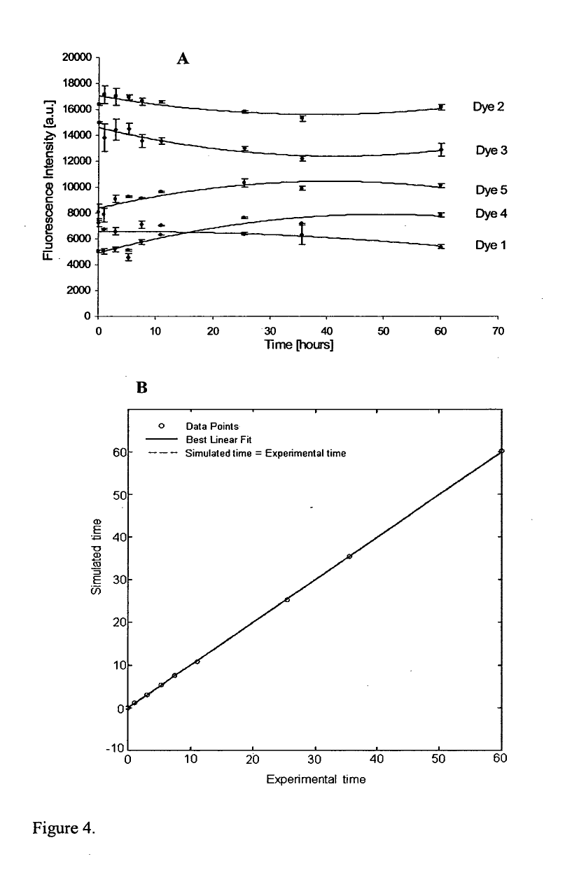

The bacterial strain used in the experiment was Escherichia coli (JM 83). Bacteria were recovered from frozen state by growing them in a Miller LB broth (solution of 12.5 g of the medium in 0.5 L of Milli-Q water) overnight at 37° C. and subcultured. The bacteria colonies were transferred into centrifuge tubes filled with 20 ml of liquid medium and incubated for 60 hours at 37° C. The tubes with bacteria cells were collected at different intervals and centrifuged at 2800 rpm for 20 min. The supernatant was filtered through a 0.22 μm filter and 3 ml of the filtrate was transferred into a quartz cuvette. 100 μl of the mixture of fluorescent dyes as used in Examples 1-3 was added to the cuvette and the fluorescence of the dyes measured as described previously(with exposure times of 0.5 s). The maximum of fluorescence intensity for each dye was plotted as a function of time of bacteria growth (FIG. 4 A). The results of simulation (prediction) of bacterial culture growth phase and the real phase of growth (monitored by measuring absorbance of cell culture at 550 nm) are shown in FIG. 4 B. The figure illustrates linear regression between actual time of bacteria growth (Targets) and the time predicted using neural network (Network Output).

### Example 5

### Identification of Cell Culture Using Mixture of Fluorescent Dyes

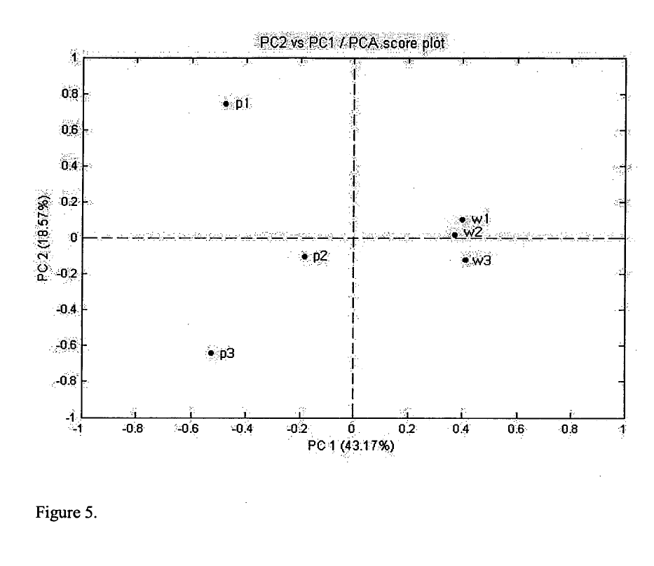

Two strains of yeast were used in the experiments: Debaryomyces Hansenii CBS. 941 and wild pink yeast (occurring naturally). The yeasts were grown in solidified agar plates containing the malt extract agar (solution of 12.5 g of the medium in 0.5 L of Milli-Q water) and 100 ppm chloramphenicol at 25° C. and subcultured until pure colonies were obtained. The yeasts were transferred into 20 ml of medium (solution of 1 g of the Yeast Extract Powder medium containing 1 g of glucose in 0.5 L of Milli-Q water) and incubated at 25° C. for 12 hours. The supernatant from two strains of yeast suspensions was taken and filtrated through a 0.22 μm filter and collected in new tubes. 3 ml of liquid were transferred into quartz cuvette and mixed with 100 μl of the mixture of fluorescent dyes. Fluorescence measurements were made using the 3D spectrofluorometer. The spectra of two strains of the yeasts were analysed using Principal Component Analysis (PCA) method. The PCA of the fluorescence spectra of white and pink strain of yeasts was made using MATLAB version 7.3.0.267 R2006b, MathWorks Inc. and the PLS Toolbox version 3.5 (Eigenvector Research Inc). The excitation-emission-intensity data were rearranged and independent variables as components found using PCA (FIG. 5). The W1-W3 points are related to white and P1-P3 to pink yeasts. The separation of clusters clearly indicates the possibility of differentiating these two different strains of cells.

### Example 6

### Simultaneous Monitoring of Multiple Parameters

This used the same dye mixture as the previous examples, and the same instrumentation. Further details are given in Table 2.

TABLE 2
List of fluorescent dyes selected for the sensor array of Example 6

| | | | | | Extinction | |
|---|---|---|---|---|---|---|
| | | | Excitation | Emission | coefficient | Supplier |
| | Dye Name | Sensitivity | [nm] | [nm] | [cm⁻¹M⁻¹] | CAS Number |
| 1 | 8-Hydroxypyrene- | pH | 470 | 527 | 24,000 | Sigma-Aldrich |
| | 1′,3,6-Trisulfonic | ionic strength | | | (water) | 6358-69-6 |
| | Acid | | | | | |
| 2 | Oregon Green 514 | pH | 504 | 528 | 86,000 | Molecular Probes |
| | carboxylic acid | | | | (DMF) | N/A |
| 3 | Rhodamine B | temperature | 541 | 576 | 88,000 | Sigma-Aldrich |
| | | | | | (water) | 81-88-9 |
| 4 | Tris (4,7-diphenyl- | oxygen | 541 | 628 | 14600 | Sigma-Aldrich |
| | 1,10-phenanthroline) | | | | (water) | 50525-27-4 |
| | ruthenium dichloride | | | | | |
| 5 | Thionin Acetate | hydrogen | 463 | 637 | 53,000 | Sigma-Aldrich |
| | | peroxide | | | (water) | 78338-22-4 |

All measurements were performed in phosphate buffers (PBS) at pH ranging from pH 5.0 to pH 8.0, temperature from 25° C. to 40° C., dissolved oxygen (DO) from 0 ppm to 21.6 ppm and PBS concentration ranging from 5 mM to 150 mM. In each measurement 3 ml of suitable buffer was transferred into 4 ml quartz cuvette and then 50 μl of the mixture of five fluorescent dyes was added. The temperature of the cuvette containing dyes solution was adjusted externally within range of 25-40° C. using thermostatic water-bath (Grant Instruments Ltd, Model 0331, Cambridge, UK). The DO concentration was controlled using oxygen probe (World Precision Instruments Ltd, OXEL-1, Stevenage, UK) and potentiostat-galvanostat (Uniscan Instrument Ltd, PG580, Buxton, UK).

ANN simulations. The spectral characteristics of dye array and changes caused by interactions with its surroundings were investigated using the ANN simulation. The development of neural network was implemented in MATLAB (version 7.3.0.267 R2006b MathWorks Inc., 2006) including the Neural Network Toolbox version 5.0.1. The prediction model used for simulation of four parameters was created based on feedforward backpropagation network. It consisted of input layer, one hidden layer and output layer. The input layer was made up of 5 neurons that corresponded to fluorescent emission of 5 fluorescent dyes (taken at wavelengths shown in Table 2) and 1 neuron in the output layer that corresponded to the value of a parameter of interest. The hidden layer consisted of 25 neurons. The network training process was performed using the Levenberg-Marquardt (LM) backpropagation training function. All calculations were executed in Windows XP (Microsoft) on a laptop with 2.2 GHz Intel Core 2 Duo processor and 2 GB of RAM.

The ability of fluorescent dyes to detect simultaneously changes of pH, temperature, DO concentration and different PBS buffer concentration was investigated. Initial experiments were performed to establish the influence of single parameter on the response spectrum of the dye array while other parameters were kept constant. Spectroscopic prosperities of each indicator were studied separately and in a mixed solution with others. We first investigated the effect on fluorescence characteristic of the multisensor caused by pH changes. Since the excitation/emission maxima of fluorescent dyes are well separated (see FIG. 1), their individual characteristics due to decreasing value of pH are easily distinguished. Fluorescence intensity of dyes changing differently depends on their sensitivity to pH. The most significant changes appear for 8-Hydroxypyrene-1′,3,6-Trisulfonic Acid (HPTS) which is specified as a near-neutral pH indicator. Emission intensities of other dyes also fluctuate, however very slightly in compare to HPTS. Most probably it is a result of interactions with molecules of other dyes and interferences caused by energy transfer or the effect of temperature results in changes of their optical and electrical properties. Following this example the response of dyes array has been recorded for other parameters tested in this study (data not shown). In all cases reasonable changes in fluorescent spectrum of dye mixture was observed in response to change of temperature, concentration of oxygen or buffer concentration. As expected, all dyes responded to some degree to the changes of each of the parameters tested. Moreover, the presence of other dyes in the same solution did not change their individual spectroscopic profile. However, the response of sensor array was not a mechanical summation of individual responses of fluorescent dyes but also the product of their interaction and cross-interferences caused by varying physico-chemical parameters. This obviously complicates the analysis especially in the case if several parameters are changing simultaneously.

To study the effect of several varying parameters on the response of dyes array, the fluorescence emission values were recorded for a number of samples each having different values of pH, temperature, concentration of oxygen and buffer concentration. The fluorescence signals were analyzed using ANN as a pattern recognition method. The aim was to check whether it is possible in these tests to quantify values of individual parameters of their surrounding media using data collected from the fluorescence response of the dyes array.

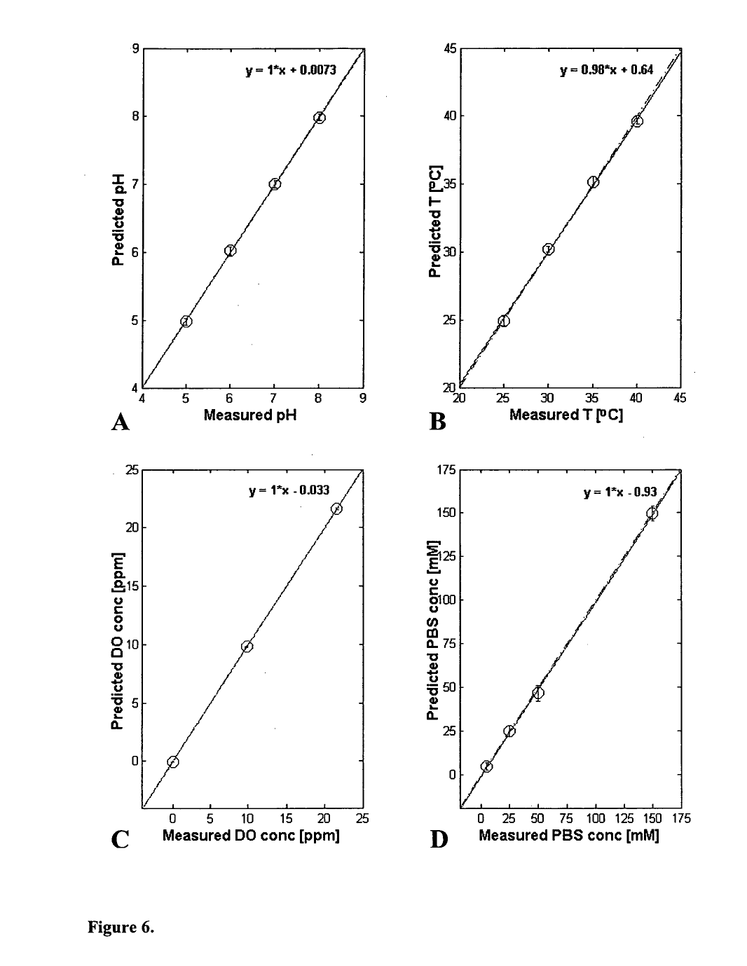

ANN simulations. The ANN was first trained using input data set consisting of fluorescence intensities of dyes array in response to presence of well defined samples. The output data set consisted of values of pH, temperature, DO and PBS concentrations. However, it was expected that using the whole fluorescent spectrum of each measurement for training of the ANN would give the best contrast in spectra due to changes of parameters, this would entail large amount of data. As each input is multiply by its connection weight it would result in drastic increase in training time of the network. To optimise this process only the maxima of fluorescence intensity of each dye were chosen to represent the profile of original spectra as actual input data for the ANN. Those points indicated wavelengths of the most significant variation in multisensor responses and were used later in training of the ANN. The training process involves adjusting internal ANN weights to minimise the error between the input data and corresponding outputs. First, it was performed on several networks contain different number of neurons in hidden layer, using different learning algorithms and varying momentum. It was done to test the ability of the ANN to detect and quantify changes of pH, temperature, DO and a buffer concentration with the smallest prediction error. As a result the network of the best performance is selected and applied later in simultaneously measurements of four parameters. This network was trained using back propagation algorithm, including define momentum and learning rate adjusted adequate to requirements of each parameter accuracy. Once the network was trained sufficiently, test sets of data (unseen samples) were used for the model evaluation. Based on its learning experience, the network was capable of prediction the output values of unknown samples. Results of ANN simulations are shown in FIG. 6. Graphs illustrate the correlation between real, measured values of parameters (X axis), and values determined by the ANN(Y axes), (FIG. 6A—pH, FIG. 6B—temperature, FIG. 6C—DO concentration and FIG. 6D—PBS concentration). Points marked as circles (◯) indicate data points of networks simulations of unseen samples. FIG. 6 shows very good accuracy of predicted values using ANN relative to measured values. Obtained results show highly accurate prediction of four parameters with the worst error of only 0.06 pH, 0.40 temperature (° C.), 0.04 DO (ppm) and 4.65 PBS concentration (mM). It proves that combination of fluorescence signals of dye array as a response to simultaneous changes of four parameters together with ANN allows simultaneously identification and quantification of compounds in studied media.

Whereas this example employed a particular dye array composed of five fluorescent dyes for the simultaneous determination of pH, temperature, dissolved oxygen and buffer concentration, the invention is applicable to different parameters or parameter sets, and different dyes and dye arrays (in terms of the nature and the number of the dyes). This optical muti-sensing system is combined with an artificial neural network for the processing of spectral responses in the same way as electrochemical sensor array known as an electronic nose. The mixture of fluorescent dyes has been design based on dyes specific sensitivity to parameters of interests with capability of generating fluorescence intensity changes in response to changes in environmental conditions. Moreover, they absorb and emit light over the VIS-NIR wavelength range, reveal good photostability and solubility in water, and also have low toxicity which is very promising for studies biological samples. Additionally, our favoured dyes are highly fluorescent, commercially available and inexpensive which reduces time and cost of measurements and analysis. Spectroscopic investigations on the dye array have proved that five fluorescent dyes can be mixed together, excited simultaneously and provide collective spectrum with well-separated emission maxima. This gives a possibility to observe individual contribution of five dyes in the whole fluorescence spectrum of their mixture. It was also found that the presence of all dyes in the mixed solution does not affect their individual activity. Although it causes some interference, each dye retains its specific sensitivity (Table 2). They behave the same as when they are separate. Therefore, by using several indicators it is possible to measure several parameters simultaneously. In this example, dye array composed of five dyes is presented but the possibility of increasing their number or replacement is always open, depending on various applications. This makes a fluorescent dye system very flexible and provides a great tool for rapid, real-time and simultaneous determination of various environmental compounds. However, the analysis of collective data starts to be more complicated while changes of some parameters (e.g. temperature) affect the spectroscopic signal of more then one dye. It brings additional fluctuation of fluorescence response signal. Temperature could still be controlled by Rhodamine B whose fluorescence was not affected by pH, oxygen or PBS concentration and is distinctly sensitive to temperature.

The complex fluorescence patterns were analysed using the ANN. This combination of response signals of the optical multisensor together with a pattern recognition method based on the ANN, has appeared to be very successful. The ANN has shown great ability in adapting and modelling the non-linear changes in the investigated system, and provides precise data extraction of measured samples. It resulted in high accuracy of simultaneously determination and quantification of pH temperature, OD and PBS concentration.

The potential of this tool is wide and diverse and can have great impact on variety of applications including food analysis, monitoring of ageing process for materials and commercial products, monitoring of local or temporal changes in the environment. The dyes mixture can also be used for monitoring of various parameters of living organisms including humans.

1. A method of monitoring a substrate comprising:
(a) providing a mixture containing a plurality of dyes each having an optical property which is sensitive to an external factor, wherein said optical properties of the different dyes are individually monitorable;
(b) enabling the mixture of dyes to interact with the substrate which provides said external factor, thereby influencing said optical properties of the dyes;
(c) monitoring said optical properties of said plurality of dyes to provide a corresponding plurality of output signals; and
(d) subjecting said plurality of output signals to a pattern recognition method, thereby to characterise the substrate or a parameter or condition thereof.

2. A method according to claim 1 wherein said optical properties are the same optical property of each dye.

3. A method according to claim 1 wherein said optical properties are properties of spectra of the dyes.

4. A method according to claim 3 wherein said spectra are selected from absorbance, transmission, reflectance, scattering and fluorescence spectra.

5. A method according to claim 3 wherein the optical properties which are monitored are maxima in the spectra of the different dyes whose wavelengths are separated by at least 10 nm.

6. A method according to claim 1 wherein the substrate comprises a solution and the plurality of dyes is added to and dissolved in the solution.

7. A method according to claim 6 wherein the substrate is a cell culture.

8. A method according to claim 1 wherein said pattern recognition method is selected from principal component analysis, multi-regression analysis, cluster analysis and artificial neural network analysis.

9. A method according to claim 8 in which said pattern recognition method is artificial neural network analysis.

10. A method according to claim 1 wherein said external factor is one or more of chemical composition, temperature, pH, ionic strength and electrical properties.

11. A method according to claim 10 in which said external factor comprises a chemical composition factor selected from presence of redox species, presence of surfactants and, oxygen content.

12. A method according to claim 1 wherein step (d) is used to determine a plurality of parameters of the substrate.

13. A method according to claim 12 wherein said substrate comprises a solution and said plurality of parameters include one or more of pH, temperature, dissolved oxygen concentration and phosphate buffer concentration.

14. A method according to claim 1 wherein the dyes are embedded in a solid matrix.

15. A method according to claim 14 wherein the solid matrix comprises a gel.
